(19) United States
(12) Patent Application Publication
(10) Pub. No.: US 2024/0321431 A1
(43) Pub. Date: Sep. 26, 2024

(54) PROCEDURE SUPPORT

(71) Applicant: Siemens Healthineers AG, Forchheim (DE)

(72) Inventors: Marcus Pfister, Bubenreuth (DE); Katharina Breininger, Erlangen (DE)

(21) Appl. No.: 18/614,711

(22) Filed: Mar. 24, 2024

(30) Foreign Application Priority Data
Mar. 24, 2023 (DE) 10 2023 202 684.8

(51) Int. Cl. G16H 30/20; A61B 5/117

(52) U.S. Cl. CPC G16H 30/20; A61B 5/117

(57) ABSTRACT

A method includes capturing time-resolved procedure data relating to a medical procedure, which includes procedure parameters and/or a device configuration and/or physiological data of an examination object and/or medical image data of the examination object. Time information relating to a target instant and a target procedure configuration for the target instant are provided by applying a trained function to input data. The input data is based on the procedure data. The target procedure configuration, including a target procedure parameter and/or a target device configuration, and the time information, designating an interval between an identified procedure event and the target instant and/or the target instant, is provided as the output data. At least one parameter of the trained function is adjusted based on a comparison of a training procedure configuration with a comparison procedure configuration and a comparison of training time information with comparison time information.

This application claims the benefit of German Patent Application No. DE 10 2023 202 684.8, filed on Mar. 24, 2023, which is hereby incorporated by reference in its entirety.

### BACKGROUND

The present embodiments relate to a computer-implemented method for procedure support, a computer-implemented method for providing a trained function, a provision unit, a medical imaging device, a training unit, and a computer program product.

Due to advancing technologies, operating techniques and development of instruments, ever more complex procedures are being carried out in a minimally invasive manner (e.g., using imaging monitoring by a C-arm X-ray device, such as an angiography system). Sequences and/or system operation may also become increasingly more complex hereby. In order to support a user (e.g., a medical operator, such as a male or female doctor), it is possible to automatically identify workflow acts and select suitable organ programs and/or system positions. Methods of this kind are known, for example, from the publications by Padoy N., “Machine and deep learning for workflow recognition during surgery,” Minimally Invasive Surgery and Allied Techniques, 2019, and by Arbogast N. et al., “Workflow Phase Detection in Fluoroscopic Images Using Convolutional Neural Networks,” Bildverarbeitung für die Medizin [Image Processing for Medicine], 2019. Common to these approaches for identifying workflow steps is the fact that the identification of the workflow steps identifies only fixed categories that have been defined in advance. One drawback is that it is not possible to identify user-specific workflow steps.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, individual procedure support of a user is enabled.

Independent of the grammatical term usage, individuals with male, female, or other gender identities are included within the term.

Methods and apparatuses for procedure support and methods and apparatuses for providing a trained function are described below. Features, advantages, and alternative embodiments of data structures and/or functions in methods and apparatuses for procedure support may be transferred, for example, to analogous data structures and/or functions in methods and apparatuses for providing a trained function. Analogous data structures may be characterized, for example, by the use of the prefix “training”. Further, the trained functions used in methods and apparatuses for procedure support may have been adjusted and/or provided (e.g., by methods and apparatuses for providing a trained function).

The present embodiments relate, in a first aspect, to a computer-implemented method for procedure support. In a first act a), time-resolved procedure data relating to a medical procedure is captured, which includes procedure parameters and/or a device configuration and/or physiological data of an examination object and/or medical image data of the examination object. In a further act b), an item of time information relating to a target instant and a target procedure configuration for the target instant is provided by applying a trained function to input data. The input data is based on the procedure data. Further, the target procedure information, including a target procedure parameter and/or a target device configuration, and the item of time information, designating an interval between an identified procedure event and the target instant and/or the target instant, are provided as the output data. In addition, at least one parameter of the trained function is adjusted based on a comparison of a training procedure configuration with a comparison procedure configuration and a comparison of an item of training time information with an item of comparison time information.

The above-described acts of the method may be partially or completely computer-implemented (e.g., by one or more processors). In addition, the above-described steps of the proposed method may be executed at least partially (e.g., completely) successively or at least partially simultaneously.

Capturing the procedure data may include receiving and/or recording the procedure data. Receiving the procedure data may include, for example, capturing and/or reading from a computer-readable data store and/or receiving from a data storage unit (e.g., a database), for example, using an interface. Further, the procedure data may be provided by a medical device (e.g., a medical imaging device) and/or a sensor.

The examination object may be, for example, a human and/or animal patient and/or an examination phantom (e.g., a vessel phantom).

The procedure may include administering a medication and/or arranging and/or moving and/or manipulating a medical object (e.g., a medical, such as diagnostic and/or surgical, instrument) and/or an implant on and/or in the examination object and/or recording medical image data of the examination object using a medical imaging device and/or treating at least one treatment region of the examination object (e.g., carrying out an ultrasound, brachytherapy, shockwave, and/or irradiation procedure).

In one embodiment, the procedure data may include a procedure parameter (e.g., a plurality of procedure parameters). The procedure parameter may include, for example, an administering rate and/or dose and/or composition of a medication. Alternatively or in addition, the procedure parameter may include a speed and/or direction of movement and/or positioning (e.g., a spatial position and/or orientation and/or pose) and/or trajectory of a medical object and/or medical device and/or of the examination object. Alternatively or in addition, the procedure data may include a device configuration. The device configuration may include one or more operating parameters of one or more medical devices (e.g., a positioning, such as an absolute or a relative positioning) and/or an acquisition parameter and/or a dose parameter. Alternatively or in addition, the procedure data may include physiological data of the examination object (e.g., a breathing rate and/or pulse rate and/or blood oxygen saturation and/or body temperature). Alternatively or in addition, the procedure data may include medical image data of the examination object. The medical image data may map the examination object (e.g., a joint or at least partially different examination region(s) of the examination object) in a two-dimensionally (2D) or three-dimensionally (3D) spatially resolved manner. Further, the medical image data may be time-resolved. The medical image data may be recorded by the same or different medical imaging devices (e.g., medical imaging modalities). The at least one medical imaging device for recording the image data of the examination object may include a medical X-ray device (e.g., a medical C-arm X-ray device and/or a cone beam computed tomography system (CBCT)) and/or a computed tomography system (CT system) and/or a magnetic resonance tomography system (MRT system) and/or a positron emission tomography system (PET system) and/or an ultrasound device. The time-resolved procedure data may map the procedure parameters and/or device configuration and/or physiological data and/or the medical image data within a capture period.

In act b), the item of time information relating to the target instant (e.g., an item of information with respect to the target instant) and the procedure configuration for the target instant are provided by applying the trained function to the input data. The trained function may be trained by a machine learning method. For example, the trained function may be a neuronal network (e.g., a convolutional neuronal network (CNN)) or a network including a convolutional layer.

The trained function maps input data to the output data. The output data may still depend, for example, on one or more parameters of the trained function. The one or more parameters of the trained function may be determined and/or adjusted by training. The determination and/or the adjustment of the one or more parameter(s) of the trained function may be based, for example, on a pair of items of training input data and associated training output data (e.g., comparison output data), with the trained function being applied to the training input data for generating training mapping data. For example, the determination and/or the adjustment may be based on a comparison of the training mapping data and the training output data (e.g., comparison output data). In general, a trainable function (e.g., a function with one or more as yet unadjusted parameters) is also referred to as a trained function. The trained function may be adjusted, for example, by Representation Learning, known, for example, from the publication by Gómez-Silva et al., “Deep Learning of Appearance Affinity for Multi-Object Tracking and Re-Identification: A Comparative View,” Electronics 2020, 9(11), 1757.

Other terms for trained functions are trained mapping rule, mapping rule with trained parameters, function with trained parameters, machine learning algorithm. One example of a trained function is an artificial neuronal network, with edge weights of the artificial neuronal network corresponding to the parameters of the trained function. Instead of the term “neuronal network,” the term “neuronal net” may also be used. For example, a trained function may also be a deep neuronal network (e.g., deep artificial neuronal network). A further example of a trained function is a “Support Vector Machine”. For example, other machine learning algorithms may also still be used as the trained function.

The trained function may be trained, for example, by a back propagation. First, training mapping data may be determined by applying the trained function to the training input data. After this, a deviation between the training mapping data and the training output data may be ascertained (e.g., the comparison output data) by applying an error function to the training mapping data and the training output data (e.g., the comparison output data). Further, at least one parameter (e.g., a weighting of the trained function) may be iteratively adjusted. As a result, the deviation between the training mapping data and the training output data (e.g., the comparison output data) may be minimized during training of the trained function.

In one embodiment, the trained function (e.g., the neuronal network) has an input layer and an output layer. The input layer may be configured for receiving input data. Further, the output layer may be configured for providing mapping data (e.g., the output data). The input layer and/or the output layer may each include a plurality of channels (e.g., neurons).

The input data of the trained function is based on the procedure data. For example, the input data of the trained function includes the procedure data. Further, the trained function provides the item of time information and the target procedure configuration as the output data. At least one parameter of the trained function is adjusted based on a comparison of a training procedure configuration with a comparison procedure configuration and a comparison of an item of training time information with an item of comparison time information. For example, the trained function may be provided by an embodiment of a method for providing a trained function, which is described in the following.

The item of time information provided as the output data of the trained function may designate an interval between the identified procedure event (e.g., automatically by the trained function) and the target instant. For example, the item of time information may provide an instant of the identified procedure event and the interval between the instant and the target instant. Alternatively or in addition, the item of time information may designate the target instant (e.g., as the absolute or relative time specification). In one embodiment, the target instant is in the present or in the future with respect to the most recent instant in each case, which is mapped in the procedure data. The target procedure configuration provided as the output data of the trained function may include a target procedure parameter (e.g., a plurality of target procedure parameters) and/or a target device configuration for the target instant. The target procedure parameter may include, for example, an administering rate and/or dose and/or composition of a medication. Alternatively or in addition, the target procedure parameter may include a speed and/or direction of movement and/or positioning (e.g., a spatial position and/or orientation and/or pose) and/or trajectory of a medical object and/or medical device and/or of the examination object. The target device configuration may include one or more operating parameters of one or more medical devices (e.g., a positioning, such as an absolute or a relative positioning) and/or an acquisition parameter and/or a dose parameter. For example, the target device configuration may include an item of positioning information and/or an acquisition parameter of a medical imaging device for recording medical image data of the examination object.

Providing the item of time information and the target procedure configuration may include storage on a computer-readable storage medium and/or display on a display unit and/or transfer to a provision unit. For example, a graphical display of the item of time information and/or the target procedure configuration may be displayed by the display unit.

Via the provision of the item of time information and the target procedure configuration, the method of the present embodiments for procedure support may enable adaptive, user- and/or procedure-specific support of the user.

In a further embodiment of the method for procedure support, acts a) and b) may be repeatedly executed until the occurrence of a termination condition.

In one embodiment, the termination condition may specify a maximum number of repetitions of acts a) to b). Alternatively or in addition, the termination condition may specify a maximum duration for repeated execution of acts a) and b) (e.g., in total). Alternatively or in addition, the termination condition may occur on completion of the procedure (e.g., in the presence of a corresponding user input of the user).

In one embodiment, procedure data may be captured repeatedly (e.g., continuously). The item of time information and the target procedure configuration may be provided by applying the trained function to the most recently captured procedure data in each case.

The embodiment may enable repeated (e.g., continuous) procedure support of the user.

In a further embodiment of the method for procedure support, a user identification may be captured. The input data of the trained function may also be based on the user identification.

In one embodiment, the user identification may be captured by an identification unit. The identification unit may include, for example, the input unit for capturing a user input having the user identification. Alternatively or in addition, the identification unit may include a sensor (e.g., an optical and/or electromagnetic and/or mechanical and/or acoustic sensor) for capturing the user identification. The sensor may be configured for biometric identification of the user (e.g., as a camera and/or vein scanner and/or fingerprint sensor and/or voice recognition unit). Alternatively or in addition, the sensor may be configured for capturing an identification (e.g., an ID card) of the user configured, for example, as an optical scanner and/or barcode scanner and/or QR code scanner and/or RFID scanner.

In one embodiment, the user identification may enable identification of the user or a specific user group. The user group may be defined, for example, using a competence-based classification of a plurality of users. The input data of the trained function may also be based on the user identification (e.g., also includes the user identification).

The embodiment may enable user-specific procedure support.

In a further embodiment of the method for procedure support, act b) may include outputting a workflow note for implementation of the target procedure configuration for the target instant.

In one embodiment, the workflow note having an item of information (e.g., an instruction) for implementation of the target procedure configuration may be output by an output unit before or at the target instant. The output unit may include, for example, a display unit and/or a loudspeaker. The workflow note may include an absolute and/or a relative item of information for implementation of the target procedure configuration for the target instant. For example, the workflow note may indicate the target procedure configuration (e.g., the target procedure parameter and/or the target device configuration) absolutely for implementation at the target instant. Alternatively or in addition, the workflow note may include an item of information for adjusting an instantaneous procedure configuration (e.g., the procedure parameters and/or the device configuration), so the target procedure configuration provided by the trained function as the output data may be implemented at the target instant.

In one embodiment, the workflow note may have a graphical display of the target procedure configuration (e.g., a graphical display of the target procedure parameter and/or the target device configuration for the target instant) and/or a graphical display of the item of time information (e.g., in text form and/or symbol form). Alternatively or in addition, the workflow note may have a sound output and/or speech output for implementation of the target procedure configuration. Alternatively or in addition, outputting of the workflow note may include pre-allocation of operating parameters of a medical device and/or of a medical object with the target procedure parameter and/or the target device configuration. In addition, the user may be prompted by the workflow note to confirm the pre-allocated operating parameters before or at the target instant.

The embodiment may enable particularly intuitive procedure support via the output of the workflow note.

In a further embodiment of the method for procedure support, the physiological data may include an ECG signal and/or a breathing signal and/or a movement signal and/or an EEG signal of the examination object. Alternatively or in addition, the device configuration may include an item of positioning information and/or an acquisition parameter of a medical imaging device for recording the medical image data of the examination object.

In one embodiment, the physiological data may include an electrocardiogram signal (ECG signal) and/or a breathing signal and/or a movement signal and/or an electroencephalography signal (EEG signal) of the examination object. The ECG signal may be provided by an ECG sensor. Further, the breathing signal may be provided by a breathing sensor. The EEG signal may be provided, for example, by an EEG sensor. Further, the movement signal may be provided by a motion sensor. Alternatively or in addition, the breathing signal and/or the movement signal may be determined using medical image data of the examination object. In one embodiment, the physiological data (e.g., the ECG signal and/or the breathing signal and/or the movement signal and/or the EEG signal) may characterize an instantaneous physiological state of the examination object.

Alternatively or in addition, the device configuration may include an item of positioning information and/or an acquisition parameter of the medical imaging device for recording the medical image data of the examination object. The item of positioning information may include an item of information relating to a spatial position and/or orientation and/or pose of the imaging device (e.g., instantaneous). Further, the acquisition parameter may include an operating parameter (e.g., a tube voltage and/or X-ray dose and/or detector configuration) and/or a recording geometry (e.g., a field of view (FOV)) of the imaging device for recording the medical image data.

The embodiment may enable a specific identification of the procedure event.

The present embodiments relate, in a second aspect, to a computer-implemented method for providing a trained function. In act t1), time-resolved training procedure data relating to a medical training procedure is captured, which includes a procedure parameter and/or a device configuration and/or physiological data of a training examination object and/or medical image data of the training examination object. In a further act t2), a user input is captured by an input unit, where the user input marks a comparison instant within the training procedure. In a further act t3), a comparison procedure configuration including the procedure parameter and/or the device configuration is identified at the comparison instant. In a further act t4), a comparison procedure event is identified by retrospective analysis of the training procedure data, starting from the comparison instant, in at least one predefined time window. An item of comparison time information is also determined, which designates an interval between the comparison procedure event and the comparison instant and/or the comparison instant. In a further act t5), an item of comparison time information relating to a comparison instant and an item of comparison procedure information relating to the comparison instant are provided by applying the trained function to the input data. The input data is based on the training procedure data before the comparison instant. The comparison procedure configuration, including a comparison procedure parameter and/or a comparison device configuration, and the item of comparison time information, designating an interval between an identified comparison procedure event and the comparison instant and/or the comparison instant, are provided as the output data. In a further act t6), at least one parameter of the trained function is adjusted based on a comparison of the training procedure configuration with the comparison procedure configuration and a comparison of the item of training time information with the item of comparison time information. In a further act t7), the trained function is provided.

Capturing the time-resolved training procedure data may include receiving and/or recording and/or simulating the time-resolved training procedure data. Receiving the training procedure data may include, for example, capturing and/or reading from a computer-readable data store and/or receiving from a data storage unit (e.g., a database). Further, the training procedure data may be provided by a medical device (e.g., a medical imaging device) and/or a sensor. The training procedure data may have, for example, all features and properties of the procedure data, which have been described in relation to the method of the present embodiments for procedure support, and vice versa. For example, the training procedure data may be procedure data. Further, the training examination object may have all features and properties of the examination object, which have been described in relation to the method of the present embodiments for procedure support, and vice versa. For example, the training examination object may be an examination object. For example, the training examination object may be the same or different than the examination object. The previously described method for providing a trained function may be carried out for a plurality of different training examination objects.

In act t2), the user input is captured by the input unit. The input unit may include, for example, a keyboard and/or a button and/or a joystick and/or a touchpad and/or a microphone (e.g., for speech recognition) and/or a camera (e.g., for gesture capture). In one embodiment, the input unit may be integrated in a display unit (e.g., as a resistive and/or capacitive input display (touchscreen)). The input unit may capture the user input and provide a corresponding signal to a provision unit. The user input may mark a comparison instant (e.g., a temporal trigger point) within the training procedure. For example, a capture instant of the user input may be identified as the comparison instant.

In act t3), the comparison procedure configuration including the procedure parameter and/or the device configuration may be identified at the comparison instant (e.g., including a comparison procedure parameter and/or a comparison device configuration using the training procedure data). The comparison procedure configuration may include the procedure parameter (e.g., the plurality of procedure parameters) and/or the device configuration, which have the training procedure data for the comparison instant. The comparison procedure configuration may be identified automatically. The comparison procedure parameter may include, for example, an administering rate and/or dose and/or composition of a medication. Alternatively or in addition, the comparison procedure parameter may include a speed and/or direction of movement and/or positioning (e.g., a spatial position and/or orientation and/or pose) and/or trajectory of a medical object and/or medical device and/or the training examination object. The comparison device configuration may include one or more operating parameters of one or more medical devices (e.g., a positioning, such as an absolute or a relative positioning) and/or an acquisition parameter and/or a dose parameter. For example, the comparison device configuration may include an item of positioning information and/or an acquisition parameter of a medical imaging device for recording medical image data of the training examination object.

In act t4), the training procedure data may be retrospectively analyzed, starting from the comparison instant, in at least one predefined time window (e.g., a plurality of predefined time windows). The retrospective analysis of the training procedure data may include a statistical analysis of changes in the training procedure data (e.g., the procedure parameter and/or the device configuration and/or the physiological data of the training examination object and/or the medical image data of the training examination object) within the at least one specified time window. The at least one predefined time window may describe a time span within the training procedure data with specified duration and temporal resolution. The retrospective analysis of the training procedure data may be limited to the at least one predefined time window. In one embodiment, the item of comparison time information designating the comparison instant may be provided. Alternatively or in addition, the interval between the comparison procedure event and the comparison instant may be identified using the identified comparison procedure event. For example, the item of comparison time information may designate an instant of the identified comparison procedure event and the interval between the instant of the identified comparison procedure event and the comparison instant.

The item of training time information relating to the training instant and the training procedure configuration for the training instant are provided by applying the trained function to the input data. The input data of the trained function is based on the training procedure data before the comparison instant (e.g., the training procedure data up to the comparison instant). For example, the input data of the trained function may include the training procedure data before the comparison instant. The trained function may provide the training procedure configuration and the item of training time information as the output data.

The at least one parameter of the trained function may be adjusted via the comparison of the training procedure configuration with the comparison procedure configuration and the comparison of the item of training time information with the item of comparison time information. The comparison may include a determination respectively of a deviation between the training procedure configuration and the comparison procedure configuration and between the item of training time information and the item of comparison time information. The at least one parameter of the trained function may be adjusted such that the deviations (e.g., a total deviation and/or the individual deviations) may be minimized, respectively. Adjusting the at least one parameter of the trained function may include optimizing (e.g., minimizing) a cost value of a cost function, with the cost function characterizing (e.g., quantifying) the deviation between training procedure configuration and the comparison procedure configuration and the deviation between the item of training time information and the item of comparison time information. For example, adjusting the at least one parameter of the trained function may include a regression of the cost value of the trained function.

Providing the trained function may include, for example, storage on a computer-readable storage medium (e.g., a non-transitory computer-readable storage medium) and/or transfer to a provision unit.

In one embodiment, a trained function may be provided by the method, and this may be used in an embodiment of the method for procedure support. Any situations (e.g., comparison procedure events) within the training procedure may be identified (e.g., marked) by the user due to capturing of the user input for marking the comparison instant. Comparable procedure events when applying the trained function to procedure data of a further procedure may be identified by adjusting the at least one parameter of the trained function.

In a further embodiment of the method for providing a trained function, acts t1) to t6) may be repeatedly executed until the occurrence of a termination condition.

In one embodiment, the termination condition may specify a maximum number of repetitions of acts t1) to t6). Alternatively or in addition, the termination condition may specify a maximum duration for repeated execution of acts t1) and t6) (e.g., in total). Alternatively or in addition, the termination condition may occur on completion of the training procedure (e.g., in the presence of a corresponding user input of the user).

In one embodiment, training procedure data may be captured repeatedly (e.g., continuously). Further, a plurality of comparison instants may be marked using the repeatedly captured user input. The comparison procedure configuration corresponding to the comparison instants in each case may be identified by the retrospective analysis of the training procedure data, starting from the respective comparison instant. Further, one item of comparison time information respectively may be determined at each of the comparison instants. The items of comparison time information and the comparison procedure configurations may be provided by applying the trained function to the most recently captured item of training procedure data in each case.

In one embodiment, acts t1) to t6) may be executed repeatedly for different training procedures and/or users and/or training examination objects.

The embodiment may enable repeated capture of comparison instants and identification of comparison procedure events using the user inputs. The user may hereby mark a plurality of (e.g., any number of comparison instants using the training procedure data). The trained function may be configured for identification of the procedure events corresponding to the comparison instants.

In a further embodiment of the method for providing a trained function, a training user identification may be captured. The input data of the trained function may also be based on the training user identification.

The training user identification may have, for example, all features and properties of the user identification that were described in relation to the method of the present embodiments for procedure support, and vice versa. For example, the training user identification may be a user identification. The training user identification may be captured analogously to capturing of the user identification. In one embodiment, the input data of the trained function may also be based on the training user identification (e.g., also include the training user identification).

In one embodiment, acts t1) to t6) may be executed repeatedly for different users. For example, user inputs of different users with different user identifications may be captured in act t2).

The embodiment may embody the trained function for user-specific provision of the item of training time information (e.g., of the item of time information) and the training procedure configuration (e.g., the target procedure configuration).

In a further embodiment of the method for providing a trained function, act t4) may include a manual identification of the comparison procedure event or a manual adjustment of an automatically identified candidate procedure event.

According to a first variant, the comparison procedure event may be identified manually (e.g., using a further user input). For this, the further user input may identify the instant of the comparison procedure event (e.g., a capture instant of the further user input may identify the instant of the comparison procedure event). Alternatively or in addition, a graphical display of the training procedure data may be displayed by a display unit. The user may identify the comparison procedure event using the graphical display of the training procedure data. For example, the further user input may be captured with respect to the graphical display, and the instant of the comparison procedure event may be identified using the further user input.

According to a second variant, first, a candidate procedure event may be identified by automatic retrospective analysis of the procedure data, starting from the comparison instant (e.g., by statistical analysis of changes in the training procedure data). An item of information relating to the candidate procedure event may be output to the user hereby, for example, as a graphical display by a display unit. The user may confirm or adjust the candidate procedure event using the further user input. For example, the user may delay the automatically identified instant of the candidate procedure event using the further user input.

A manual or semi-automatic identification of the comparison procedure event may be enabled hereby.

In a further embodiment of the method for providing a trained function, the training procedure event may be identified using a temporal change (e.g., a variance and/or standard deviation) in the training procedure data.

In one embodiment, the retrospective analysis of the training procedure data may include determining a temporal change (e.g., a variance and/or standard deviation) in the at least one predefined time window. The temporal change of individual portions of the training procedure data (e.g., the comparison procedure parameter and/or the comparison device configuration and/or the physiological data and/or the medical image data) and/or the temporal change in the procedure data may be examined in total. The instant of the comparison procedure event may be identified using an attainment or overshooting of a specified threshold value by the temporal change (e.g., the variance and/or standard deviation) of the training procedure data.

The embodiment may enable a robust identification of the comparison procedure event using the training procedure data. The trained function may be configured particularly robustly hereby.

In a further embodiment of the method for providing a trained function, the training procedure data may be retrospectively analyzed for identification of the comparison procedure event, starting from the comparison instant, in a plurality of predefined time windows with an at least partially different temporal resolution.

In one embodiment, the plurality of predefined time windows may have an at least partially (e.g., completely) different temporal resolution. The plurality of predefined time windows may have a linear or non-linear (e.g., logarithmic) temporal resolution. The plurality of predefined time windows may have identical or different window widths. The plurality of predefined time windows may be at least partially different (e.g., partially overlap), adjoin respectively at one further time window at least, or be arranged spaced apart. In one embodiment, the plurality of time windows may be retrospectively consecutively arranged, starting from the comparison instant. The training procedure data may be retrospectively analyzed, starting from the comparison instant, in the consecutive time windows. As soon as the comparison procedure event has been identified, the retrospective analysis may be stopped.

The embodiment may enable time-and/or computing-efficient and/or reliable identification of the comparison procedure event.

In a further embodiment of the method for providing a trained function, the training procedure data may be retrospectively analyzed for identification of the comparison procedure event, starting from the comparison instant, in a plurality of predefined time windows of different window width.

In one embodiment, the plurality of predefined time windows may have an at least partially (e.g., completely) different window width. In addition, the plurality of predefined time windows may have an identical or different temporal resolution. The plurality of predefined time windows may be at least partially different (e.g., partially overlap), adjoin respectively at one further time window at least, or be arranged spaced apart. In one embodiment, the plurality of time windows may be retrospectively consecutively arranged, starting from the comparison instant. The window width may increase as the interval from the comparison instant increases. A number of input values of the trained function (e.g., a number of data points of the training procedure data) may be increased hereby in order to retrospectively analyze a longer period before the comparison instant. Further, the temporal resolution of the time window may decrease as the interval from the comparison instant increases. The training procedure data may be retrospectively analyzed, starting from the comparison instant, in the consecutive time windows. As soon as the comparison procedure event has been identified, the retrospective analysis may be stopped.

The embodiment may enable a time-and/or computing-efficient and/or reliable identification of the comparison procedure event.

In a further embodiment of the method for providing a trained function, the physiological data may include an ECG signal and/or a breathing signal and/or a movement signal and/or an EEG signal of the training examination object. Alternatively or in addition, the comparison device configuration may include an item of positioning information and/or an acquisition parameter of a medical imaging device for recording medical image data of the training examination object.

In one embodiment, the physiological data may include an electrocardiogram signal (ECG signal) and/or a breathing signal and/or a movement signal and/or an electroencephalography signal (EEG signal) of the training examination object. The ECG signal may be provided by an ECG sensor. Further, the breathing signal may be provided by a breathing sensor. The EEG signal may be provided, for example, by an EEG sensor. Further, the movement signal may be provided by a motion sensor. Alternatively or in addition, the breathing signal and/or the movement signal may be determined using medical image data of the training examination object. In one embodiment, the physiological data (e.g., the ECG signal and/or the breathing signal and/or the movement signal and/or the EEG signal) may characterize an instantaneous physiological state of the training examination object.

Alternatively or in addition, the device configuration may include an item of positioning information and/or an acquisition parameter of a medical imaging device for recording the medical image data of the training examination object. The item of positioning information may include an item of information relating to a spatial position and/or orientation and/or pose of the imaging device (e.g., instantaneous). Further, the acquisition parameter may include an operating parameter (e.g., a tube voltage and/or X-ray dose and/or detector configuration) and/or a recording geometry (e.g., a field of view (FOV)) of the imaging device for recording the medical image data.

The embodiment may enable a robust identification of the comparison procedure event using the training procedure data.

According to a further embodiment, further comparison instants, and therewith corresponding comparison procedure events, comparison procedure configurations, and items of comparison time information, may be identified by an automatic analysis of the training procedure data. The automatically identified comparison instants may be proposed to the user for manual confirmation and/or adjustment (e.g., by a graphical display). The embodiment may automatically identify characteristic situations in a procedure (e.g., an operation) and subsequently propose the characteristic situations to the user (e.g., in a postprocedural analysis, such as a postoperative analysis) as possible trigger points within the training procedure.

The present embodiments relate, in a third aspect, to a provision unit that is configured for carrying out a method of the present embodiments for procedure support.

The provision unit may include a computing unit (e.g., including one or more processors), a memory unit, and/or an interface. The provision unit may be configured for carrying out a method of the present embodiments for procedure support in which the interface, the computing unit, and/or the memory unit are configured for executing the corresponding method acts.

The advantages of the provision unit substantially match the advantages of the proposed method for procedure support. Features, advantages, or alternative embodiments mentioned in this connection may similarly also be transferred to the other claimed subject matters, and vice versa.

The present embodiments relate, in a fourth aspect, to a medical imaging device, including a provision unit of the present embodiments. The medical imaging device is configured for recording medical image data of the examination object. Further, the medical imaging device is configured for providing the image data and/or a device configuration as the procedure data.

The advantages of the imaging device of the present embodiments substantially match the advantages of the method for procedure support of the present embodiments. Features, advantages, or alternative embodiments mentioned in this connection may similarly also be transferred to the other claimed subject matters, and vice versa.

The medical imaging device for recording the image data of the examination object may include a medical X-ray device (e.g., a medical C-arm X-ray device and/or a cone beam computed tomography system (CT, CBCT)) and/or a computed tomography system (CT system) and/or a magnetic resonance tomography system (MRT system) and/or a positron emission tomography system (PET system) and/or an ultrasound device.

The present embodiments relate, in a fifth aspect, to a training unit that is configured for carrying out a method for providing a trained function of the present embodiments.

The training unit may include a training interface, a training memory unit, and/or a training computing unit. The training unit may be configured for carrying out a method for providing a trained function in that the training interface, the training memory unit, and/or the training computing unit are configured to execute the corresponding method acts. For example, the training interface may be configured to execute acts t1), t2), and/or t7). Further, the training computing unit and/or the training memory unit may be configured to execute acts t3) to t6).

The advantages of the training unit substantially match the advantages of the method for providing a trained function of the present embodiments. Features, advantages, or alternative embodiments mentioned in this connection may similarly also be transferred to the other subject matters, and vice versa.

The present embodiments relate, in a sixth aspect, to a computer program product with a computer program that may be loaded directly into a memory of a provision unit, with program segments in order to execute all acts of the method for procedure support and/or one of its aspects when the program segments are executed by the provision unit, and/or may be loaded directly into a training memory of a training unit, with program segments in order to execute all acts of a method of the present embodiments for providing a trained function and/or one of its aspects when the program segments are executed by the training unit.

The present embodiments may also relate to a computer program or computer-readable storage medium, including a trained function, that was provided by a method of the present embodiments or one of its aspects.

An implementation largely in terms of software has the advantage that even previously used provision units and/or training units may easily be retrofitted via a software update in order to work according to the present embodiments. Apart from the computer program, a computer program product of this kind may optionally include additional component parts, such as documentation and/or additional components, and hardware components, such as hardware keys (e.g., dongles, etc.), in order to use the software.

### BRIEF DESCRIPTION OF THE DRAWINGS

Example embodiments of the invention are represented in the drawings and will be described in more detail below. Same reference numerals will be used in the different figures for same features. In the drawings:

FIGS. 1 to 3 show schematic representations of different embodiments of a computer-implemented method for procedure support;

FIG. 4 shows a schematic representation of an embodiment of a computer-implemented method for providing a trained function;

FIG. 5 shows a schematic representation of training procedure data and comparison procedure events of a training procedure;

FIGS. 6 and 7 show schematic representations of different embodiments of a computer-implemented method for providing a trained function;

FIG. 8 shows a schematic representation of training procedure data and comparison procedure events of different training procedures;

FIG. 9 shows a schematic representation of a provision unit;

FIG. 10 shows a schematic representation of a training unit;

FIG. 11 shows a schematic representation of a medical imaging device including a provision unit; and

FIG. 12 shows a schematic representation of an example of a user interface for capturing a user input.

### DETAILED DESCRIPTION

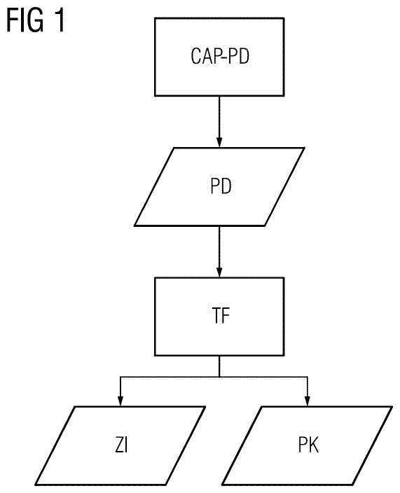

FIG. 1 schematically represents an embodiment of a computer-implemented method for procedure support. In a first act a), time-resolved procedure data PD relating to a medical procedure may be captured CAP-PD, which includes procedure parameters and/or a device configuration and/or physiological data of an examination object and/or medical image data of the examination object. The physiological data may include an ECG signal and/or a breathing signal and/or a movement signal and/or an EEG signal of the examination object. Further, the device configuration may include an item of positioning information and/or an acquisition parameter of a medical imaging device for recording the medical image data of the examination object. In a further act b), an item of time information ZI relating to a target instant and a target procedure configuration PK for the target instant may be provided by applying a trained function TF to input data. The input data may be based on the procedure data PD. Further, the target procedure configuration PK, including a target procedure parameter and/or a target device configuration, and the item of time information ZI, designating an interval between an identified procedure event and the target instant and/or the target instant, may be provided as output data.

In one embodiment, act b) may include outputting a workflow note for implementation of the target procedure configuration PK for the target instant.

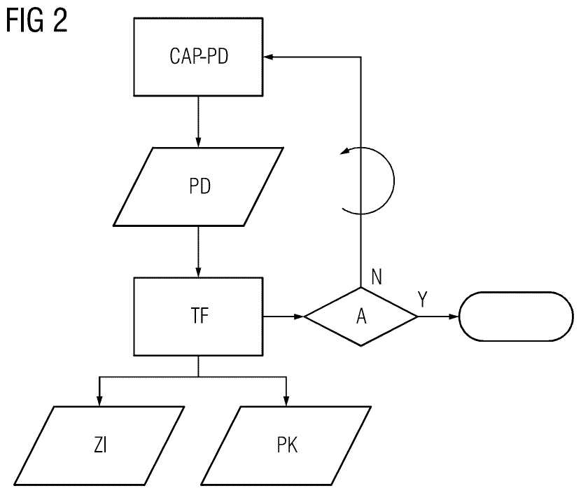

FIG. 2 shows a schematic representation of a further embodiment of the method for procedure support. Acts a) and b) may be executed repeatedly until the occurrence Y of a termination condition A (e.g., in the case of non-occurrence N of the termination condition A).

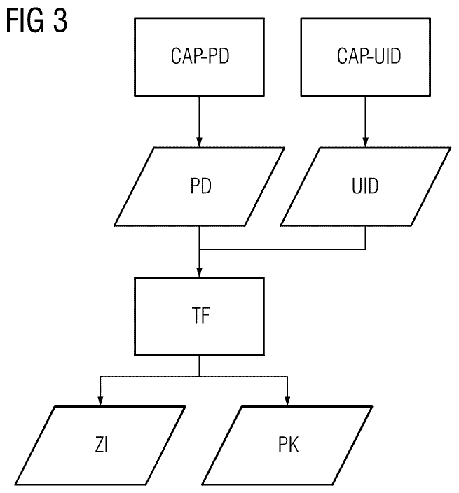

FIG. 3 shows a schematic representation of a further embodiment of the method for procedure support. A user identification UID may be captured CAP-UID. The input data of the trained function TF may also be based on the user identification UID.

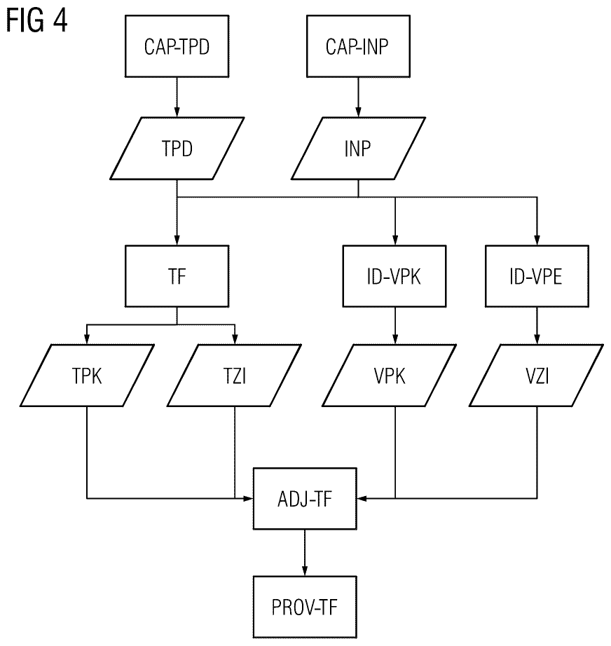

FIG. 4 shows a schematic representation of an embodiment of a computer-implemented method for providing a trained function PROV-TF. In act t1), time-resolved training procedure data TPD relating to a medical training procedure may be captured CAP-TPD, which includes a procedure parameter and/or a device configuration and/or physiological data of a training examination object and/or medical image data of the training examination object. In one embodiment, the physiological data may include an ECG signal and/or a breathing signal and/or a movement signal and/or an EEG signal of the training examination object. Further, the comparison device configuration may include an item of positioning information and/or an acquisition parameter of a medical imaging device for recording the medical image data of the training examination object.

In a further act t2), a user input INP may be captured CAP-INP by an input unit. The user input INP may mark a comparison instant within the training procedure. In a further act t3), a comparison procedure configuration VPK, including the procedure parameter and/or the device configuration at the comparison instant, may be identified. In a further act t4), a comparison procedure event may be identified ID-VPE by retrospective analysis of the training procedure data TPD, starting from the training instant, in at least one predefined time window. Further, an item of comparison time information VZI may be determined. The item of comparison time information VZI designates an interval between the comparison procedure event and the comparison instant and/or the comparison instant. In one embodiment, act t4) may include a manual identification of the comparison procedure event VPE or a manual adjustment of an automatically identified candidate procedure event. Alternatively or in addition, the comparison procedure event VPE may be identified ID-VPE using a temporal change (e.g., a variance and/or standard deviation) in the training procedure data TPD.

In a further act t5), an item of training time information TZI relating to a training instant and a training procedure configuration TPK for the training instant may be provided by applying the trained function TF to input data. The input data may be based on the training procedure data TPD before the comparison instant. In addition, the training procedure configuration TPK, including a training procedure parameter and/or a training device configuration, and the item of training time information TZI, designating an interval between an identified training procedure event and the training instant and/or the training instant, may be provided as the output data. In a further act t6), at least one parameter of the trained function TF may be adjusted based on a comparison of the training procedure configuration TPK with the comparison procedure configuration VPK and a comparison of the item of training time information TZI with the item of comparison time information VZI. In a further act t7), the trained function TF may be provided PROV-TF. The trained function TF may also be regarded as an analysis unit for the training procedure data TPD (e.g., the procedure data PD).

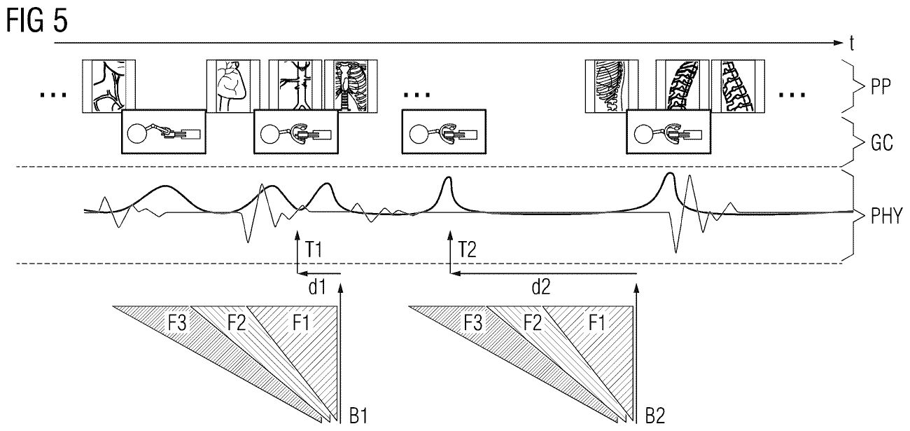

FIG. 5 shows a schematic representation of training procedure data TPD and comparison procedure events VPE of an example training procedure. The training procedure data TPD may include time-resolved medical image data PP of the examination object, a device configuration GC (e.g., positionings of a medical C-arm X-ray device for recording the medical image data PP), procedure parameters (not shown here), and physiological data PHY of the examination object. In one embodiment, the comparison instants B1 and B2 may be identified (e.g., as bookmarks) using the user input INP during the repeated execution of acts t1) to t6). The training procedure data TPD may be retrospectively analyzed ID-VPE for identification of the comparison procedure events VPE, starting from the respective comparison instant B1 and B2, in a plurality of predefined time windows (e.g., periods, F1, F2 and F3 with at least partially different temporal resolution). The retrospective analysis of the training procedure data TPD in the plurality of predefined time windows F1, F2 and F3 with at least partially different temporal resolution may be regarded as a multi-resolution approach. In addition, the plurality of predefined time windows F1 to F3 may have different window widths.

Starting from the first comparison instant B1, a comparison procedure event, for example, may be identified ID-VPE at the instant T1 within the first window width F1. The item of comparison time information VZI may be determined for this, which designates the interval dl between the first comparison procedure event and the first comparison instant B1 and/or the comparison instant B1. Starting from the second comparison instant B2, a further comparison procedure event, for example, may be identified ID-VPE at the instant T2 within the third window width F3. The item of comparison time information VZI may be determined for this, which designates the interval d2 between the further comparison procedure event and the second comparison instant B2 and/or the comparison instant B2.

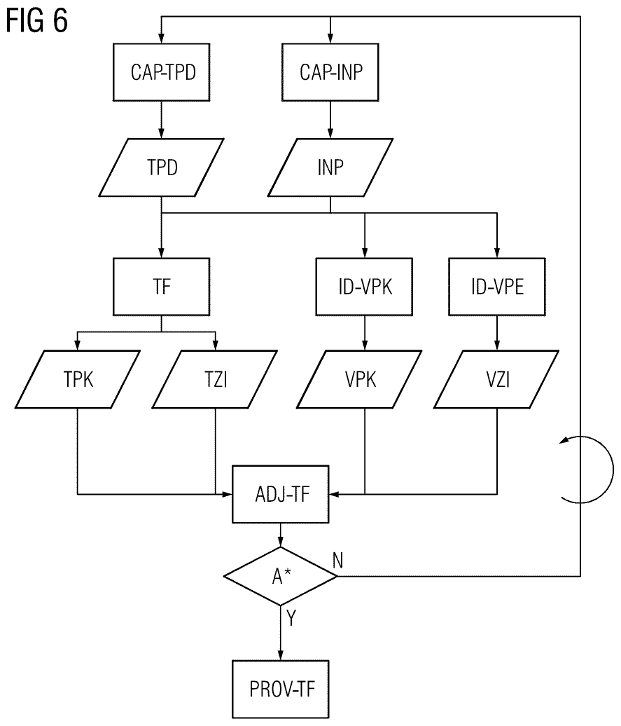

FIG. 6 shows a schematic representation of a further embodiment of the method for providing a trained function PROV-TF. Acts t1) to t6) may be repeatedly executed until the occurrence Y of a termination condition A*.

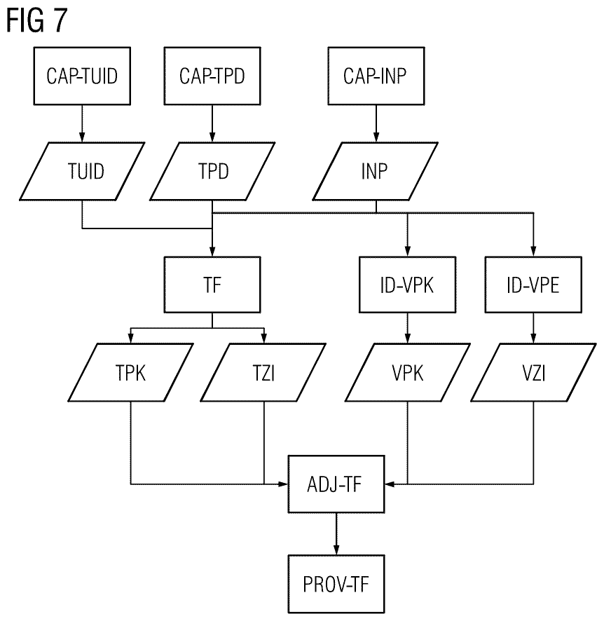

FIG. 7 shows a schematic representation of a further embodiment of the method for providing a trained function PROV-TF. A training user identification TUID may be captured CAP-TUID. Further, the input data of the trained function TF may also be based on the training user identification TUID.

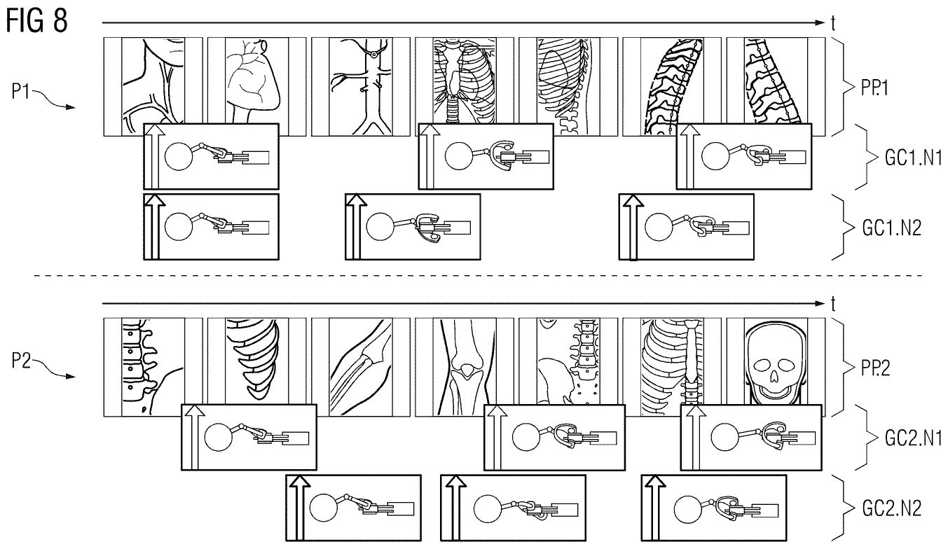

FIG. 8 shows a schematic representation of training procedure data TPD and comparison procedure events VPE of different training procedures P1 and P2. In one embodiment, first time-resolved training procedure data, including first image data PP.1 and first procedure parameters (not shown here), relating to the first training procedure may be captured. The first training procedure data may also include a first device configuration GC1.N1 for the first training procedure of a first user and a second device configuration GC1.N2 for the first training procedure of a second user. In addition, second time-resolved training procedure data, including second image data PP.2 and second procedure parameters (not shown here), relating to the second training procedure may be captured. The second training procedure data may also include a second device configuration GC2.N1 for the second training procedure of the first user and a second device configuration GC2.N2 for the second training procedure of the second user.

In one embodiment, the training user identification TUID of the first and second users may be captured CAP-TUID. In addition, the input data of the trained function TF may also be based on the training user identification TUID. In addition, the user input INP for marking the comparison instants of the first user and the second user may be captured CAP-INP. A user-specific identification of the comparison procedure configuration and the comparison procedure events may be enabled hereby.

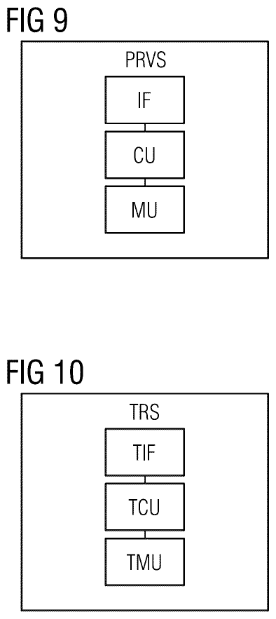

FIG. 9 shows a schematic representation of one embodiment of a provision unit PRVS. The provision unit PRVS may include a computing unit CU, a memory unit MU, and/or an interface IF. The provision unit PRVS may be configured for carrying out a method of the present embodiments for procedure support in which the interface IF, the computing unit CU, and/or the memory unit MU are configured to execute the corresponding method acts.

FIG. 10 shows a schematic representation of one embodiment of a training unit TRS. The training unit TRS may include a training interface TIF, a training memory unit TMU, and/or a training computing unit TCU. The training unit TRS may be configured for carrying out a method for providing a trained function PROV-TF in that the training interface TIF, the training memory unit TMU, and/or the training computing unit TCU are configured to execute the corresponding method acts. For example, the training interface TIF may be configured to execute acts t1), t2), and/or t7). Further, the training computing unit TCU and/or the training memory unit TMU may be configured to execute acts t3) to t6).

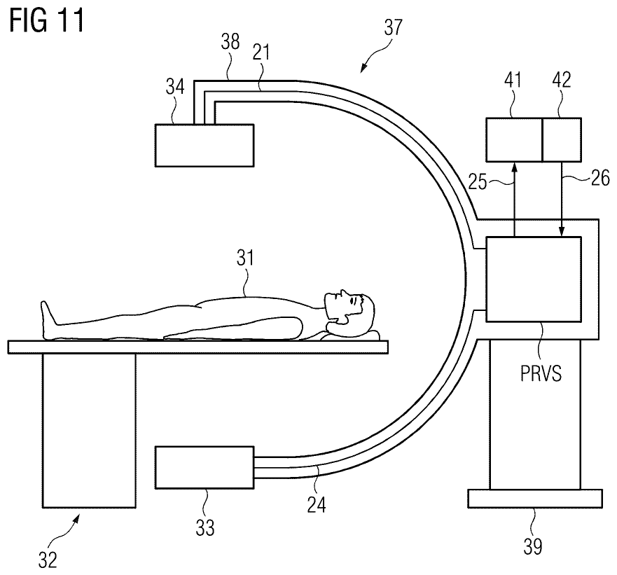

FIG. 11 shows, by way of example, for a medical imaging device, a schematic representation of a medical C-arm X-ray device 37, including a provision unit PRVS of the present embodiments. The medical C-arm X-ray device 37 may, for example, have a detector 34 (e.g., an X-ray detector) and a source 33 (e.g., an X-ray source) that are arranged on a C-arm 38 in a defined arrangement. The C-arm 38 of the C-arm X-ray device 37 may be mounted to as to move about one or more axes. Further, the C-arm X-ray device 37 may include a movement unit 39 (e.g., a wheel system and/or a robot arm and/or a rail system) that enables a movement of the C-arm X-ray device in the space. For recording medical image data of the examination object 31, positioned on a patient supporting apparatus 32, the provision unit PRVS may send a signal 24 to the X-ray source 33. The X-ray source 33 may then emit an X-ray beam bundle. When the X-ray beam bundle strikes a surface of the detector 34, after an interaction with the examination object 31, the detector 34 may send a signal 21 to the provision unit PRVS. The provision unit PRVS may capture the image data using the signal 21. The medical C-arm X-ray device 37 may also provide the image data and/or a device configuration (e.g., a positioning and/or an acquisition parameter and/or a dose parameter) as the procedure data PD.

The imaging device may also have an input unit 42 (e.g., a keyboard) and a display unit 41 (e.g., a monitor and/or a display and/or a projector). The input unit 42 may be integrated in the display unit 41 (e.g., in the case of a capacitive and/or resistive input display). The input unit 42 may be configured for capturing the user input CAP-INP. For this, the input unit 42 may send, for example, a signal 26 to the provision unit PRVS. The provision unit PRVS may be configured to identify the training instant using the user input.

The display unit 41 may be configured to display a graphical display of the procedure data PD and/or the procedure configuration and/or the item of time information and/or the workflow note. The provision unit PRVS may a send a signal 25 to the display unit 41 for this purpose.

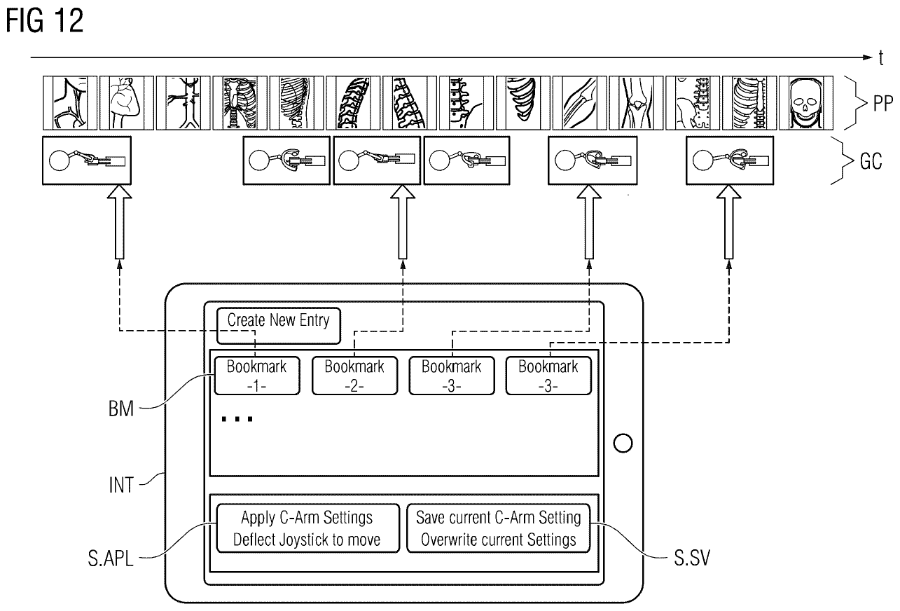

FIG. 12 shows a schematic representation of an example of a user interface INT for capturing a user input CAP-INP. In one embodiment, a graphical display of the training procedure data TPD may be displayed for the user by a display unit. For example, a graphical display of the time-resolved image data PP and/or the device configuration GC may be displayed. The user interface INT may be an input menu of an input unit for capturing the user input INP. The user may mark the comparison instant using a pushbutton BM in the user interface INT. In addition, the instantaneous comparison procedure configuration VPK may be stored by actuation of the pushbutton S.SV by the user.

The user interface INT may also be used in a method for procedure support. Providing the target procedure configuration may include proposing and/or pre-allocating the instantaneous procedure parameters and/or device configuration with the target procedure parameter and/or the target device configuration. By actuating the pushbutton S.APL, for example, the user may accept (e.g., apply) the proposed and/or pre-allocated target procedure parameters and/or target device configuration.

The schematic representations contained in the described figures do not depict any kind of scale or size ratios.

To conclude, reference is again made to the fact that the methods described in detail above and the represented apparatuses are merely example embodiments that a person skilled in the art may modify in various ways without departing from the scope of the invention. Further, use of the indefinite article “a” or “an” does not preclude the relevant features from also being present a number of times. Similarly, the terms “unit” and “element” do not preclude the relevant components from being composed of a plurality of cooperating sub-components that may optionally also be spatially distributed.

In the context of the present application, the expression “on the basis of” may be understood, for example, within the meaning of the expression “using”. For example, wording, according to which a first feature is generated (e.g., alternatively, ascertained, determined, etc.) based on a second feature, does not preclude the first feature from being generated (e.g., alternatively, ascertained, determined, etc.) based on a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for procedure support, the method being computer-implemented and comprising:
capturing time-resolved procedure data relating to a medical procedure, the time-resolved procedure data comprising procedure parameters, a device configuration, physiological data of an examination object, medical image data of the examination object, or any combination thereof; and
providing an item of time information relating to a target instant and a target procedure configuration for the target instant, the providing comprising applying a trained function to input data, wherein the input data is based on the time-resolved procedure data,
wherein the target procedure configuration, comprising a target procedure parameter, a target device configuration, or the target procedure parameter and the target device configuration, and the item of time information, designating an interval between an identified procedure event and the target instant, the target instant, or the interval and the target instant, is provided as output data, and
wherein at least one parameter of the trained function is adjusted based on a comparison of a training procedure configuration with a comparison procedure configuration and a comparison of an item of training time information with an item of comparison time information.

2. The method of claim 1, wherein the capturing and the providing are repeatedly executed until occurrence of a termination condition.

3. The method of claim 1, further comprising capturing a user identification,
wherein the input data of the trained function is also based on the user identification.

4. The method of claim 1, wherein the providing comprises outputting a workflow note for implementation of the target procedure configuration for the target instant.

5. The method of claim 1, wherein:
the physiological data comprises an ECG signal, a breathing signal, a movement signal, an EEG signal, or any combination thereof of the examination object;
the device configuration comprises an item of positioning information, an acquisition parameter of a medical imaging device for recording the medical image data of the examination object, or a combination thereof; or
a combination thereof.

6. A method for providing a trained function, the method being computer-implemented and comprising:
capturing time-resolved training procedure data relating to a medical training procedure, the time-resolved training procedure data comprising a procedure parameter, a device configuration, physiological data of a training examination object, medical image data of the training examination object, or any combination thereof;
capturing a user input using an input unit, wherein the user input marks a comparison instant within the medical training procedure;
identifying a comparison procedure configuration comprising the procedure parameter, the device configuration, or the procedure parameter and the device configuration at the comparison instant;
identifying a comparison procedure event by retrospective analysis of the training procedure data, starting from the training instant, in at least one predefined time window, wherein an item of comparison time information is determined, the item of comparison time information designating an interval between the comparison procedure event and the comparison instant, the comparison instant, or the interval and the comparison instant;
providing an item of training time information relating to a training instant and a training procedure configuration for the training instant, the providing comprising applying the trained function to input data, wherein the input data is based on the training procedure data before the comparison instant, and wherein the training procedure configuration, comprising a training procedure parameter, a training device configuration, or the training procedure parameter and the training device configuration, and the item of training time information, designating an interval between an identified training procedure event and the training instant, the training instant, or the interval between the identified training procedure event and the training instant and the training instant are provided as the output data;
adjusting at least one parameter of the trained function based on a comparison of the training procedure configuration with the comparison procedure configuration and a comparison of the item of training time information with the item of comparison time information; and
providing the trained function.

7. The method of claim 6, wherein the capturing of the time-resolved training procedure data, the capturing of the user input, the identifying of the comparison procedure configuration, the identifying of the comparison procedure event, the providing of the item of training time information, and the providing of the trained function are repeatedly executed until occurrence of a termination condition.

8. The method of claim 6, further comprising capturing a training user identification, wherein the input data of the trained function is also based on the training user identification.

9. The method of claim 6, wherein the identifying of the comparison procedure event comprises a manual identification of the comparison procedure event or a manual adjustment of an automatically identified candidate procedure event.

10. The method of claim 6, wherein the comparison procedure event is identified using a temporal change of the training procedure data.

11. The method of claim 10, wherein the temporal change includes a variance, a standard deviation, or the variance and the standard deviation.

12. The method of claim 6, wherein the training procedure data is retrospectively analyzed for identification of the comparison procedure event, starting from the comparison instant, in a plurality of predefined time windows with at least partially different temporal resolution.

13. The method of claim 6, wherein the training procedure data is retrospectively analyzed for identification of the comparison procedure event, starting from the comparison instant, in a plurality of predefined time windows of different window width.

14. The method of claim 6, wherein:
the physiological data comprises an ECG signal, a breathing signal, a movement signal, an EEG signal of the training examination object, or any combination thereof;
the device configuration comprises an item of positioning information, an acquisition parameter of a medical imaging device for recording the medical image data of the training examination object, or a combination thereof; or
a combination thereof.

15. A provision unit comprising:
a processor configured for procedure support, the processor being configured for procedure support comprising the processor being configured to:
capture time-resolved procedure data relating to a medical procedure, the time-resolved procedure data comprising procedure parameters, a device configuration, physiological data of an examination object, medical image data of the examination object, or any combination thereof; and
provide an item of time information relating to a target instant and a target procedure configuration for the target instant, the providing comprising applying a trained function to input data, wherein the input data is based on the time-resolved procedure data,
wherein the target procedure configuration, comprising a target procedure parameter, a target device configuration, or the target procedure parameter and the target device configuration, and the item of time information, designating an interval between an identified procedure event and the target instant, the target instant, or the interval and the target instant, is provided as output data, and
wherein at least one parameter of the trained function is adjusted based on a comparison of a training procedure configuration with a comparison procedure configuration and a comparison of an item of training time information with an item of comparison time information.

16. A medical imaging device comprising:
a provision unit comprising:
a processor configured for procedure support, the processor being configured for procedure support comprising the processor being configured to:
capture time-resolved procedure data relating to a medical procedure, the time-resolved procedure data comprising procedure parameters, a device configuration, physiological data of an examination object, medical image data of the examination object, or any combination thereof; and
provide an item of time information relating to a target instant and a target procedure configuration for the target instant, the providing comprising applying a trained function to input data, wherein the input data is based on the time-resolved procedure data,
wherein the target procedure configuration, comprising a target procedure parameter, a target device configuration, or the target procedure parameter and the target device configuration, and the item of time information, designating an interval between an identified procedure event and the target instant, the target instant, or the interval and the target instant, is provided as output data,
wherein at least one parameter of the trained function is adjusted based on a comparison of a training procedure configuration with a comparison procedure configuration and a comparison of an item of training time information with an item of comparison time information,
wherein the medical imaging device is configured for recording medical image data of the examination object, and
wherein the medical imaging device is configured for providing the image data, a device configuration, or the image data and the device configuration as the procedure data.

17. A training unit comprising:
a processor configured for providing a trained function, the processor being configured for providing the trained function comprising the processor being configured to:
capture time-resolved training procedure data relating to a medical training procedure, the time-resolved training procedure data comprising a procedure parameter, a device configuration, physiological data of a training examination object, medical image data of the training examination object, or any combination thereof;
capture a user input using an input unit, wherein the user input marks a comparison instant within the medical training procedure;
identify a comparison procedure configuration comprising the procedure parameter, the device configuration, or the procedure parameter and the device configuration at the comparison instant;
identify a comparison procedure event by retrospective analysis of the training procedure data, starting from the training instant, in at least one predefined time window, wherein an item of comparison time information is determined, the item of comparison time information designating an interval between the comparison procedure event and the comparison instant, the comparison instant, or the interval and the comparison instant;
provide an item of training time information relating to a training instant and a training procedure configuration for the training instant, the providing comprising applying the trained function to input data, wherein the input data is based on the training procedure data before the comparison instant, and wherein the training procedure configuration, comprising a training procedure parameter, a training device configuration, or the training procedure parameter and the training device configuration, and the item of training time information, designating an interval between an identified training procedure event and the training instant, the training instant, or the interval between the identified training procedure event and the training instant and the training instant are provided as the output data;
adjust at least one parameter of the trained function based on a comparison of the training procedure configuration with the comparison procedure configuration and a comparison of the item of training time information with the item of comparison time information; and
provide the trained function.
